(19) United States
(12) Patent Application Publication
(10) Pub. No.: US 2021/0027863 A1
(43) Pub. Date: Jan. 28, 2021

(54) SEARCH TOOL FOR KNOWLEDGE DISCOVERY

(71) Applicant: BENEVOLENTAI TECHNOLOGY LIMITED, London (GB)

(72) Inventor: Daniel Paul SMITH, London (GB)

(73) Assignee: BENEVOLENTAI TECHNOLOGY LIMITED, London (GB)

(21) Appl. No.: 17/041,536

(22) PCT Filed: Mar. 28, 2019
(86) PCT No.: PCT/GB2019/050889

§ 371 Date: Sep. 25, 2020

(30) Foreign Application Priority Data
Mar. 28, 2018 (GB) 1805074.0

(51) Int. Cl. G16B 45/00; G16H 50/20; G16H 50/70

(52) U.S. Cl. CPC G16B 45/00; G16C 20/50; G16H 50/70; G16H 50/20

(57) ABSTRACT

A system is disclosed for searching a set of biological entities. The system comprises: a user input module configured to receive a user input comprising a representation of a biological entity; a search module configured to determine which entities of a set of biological entities are associated with the user input; a visualisation module configured to render a visualisation of multiple biological entities of the set and of parent-child relationships between them; and an overlay module configured to render an association indicator visually indicating one or more biological entities of the visualisation that are associated with the user input.

The present application relates to a system and computer-implemented method for performing searches and for visually indicating search results to support a user in knowledge discovery activities.

### BACKGROUND

Knowledge discoverers in a range of fields are interested in deciphering new information from the available set of knowledge. Search engines provide a powerful information retrieval tool and are ideal for retrieving established facts and information from the public domain and other information sources. Typically, search results are presented in an ordered list in order of relevance, where the relevance is calculated using a searching algorithm. Results considered to be the most relevant are presented at the top of the list and results considered to be less relevant are presented further down.

It is not uncommon for search engines to generate tens or hundreds of pages of search results. This creates a problem of information overload for the user, and the user has limited ways of efficiently sifting through or filtering the results in a way that is meaningful.

The order of relevance calculated by the searching algorithm dominates the user's way of managing and interacting with the results, and it is difficult for the user to detect patterns or trends that may be lurking in the pages of results. For example, it is very time-consuming for a user to find a significant result if it appears on page 100 of the search results. It is also difficult for a user to spot that a result on page 100 may be related to a result on page 204 in a potentially interesting way.

This presents a challenge for knowledge discoverers who are trying to discern previously unknown information such as patterns, trends and relationships from the available facts. For example, in the field of drug discovery, a drug discoverer may use a search engine to search for diseases that are related to a particular gene. All the diseases that are well-known as being associated with this gene are likely to be listed as being highly relevant at the top of the list of search results. If there is a small number of diseases that have an association with the gene but are not determined by the searching algorithm to be highly relevant, then these diseases are likely to appear further down the list, making it less likely that the drug discoverer will find them. Furthermore, if two diseases appearing far down the list are related to each other in a potentially interesting way, this is very difficult for the drug discoverer to find, especially if they are spread out for example across pages 10, 204 and 506.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter.

The present disclosure provides a system and method of searching a set of entities, for example biological entities such as diseases. A visual map of the entities—preferably a full set of the entities such as a complete set of all known human diseases—is displayed to a user together with a visual indication of which of the displayed entities are associated with a searching term. For example, if a map of diseases is displayed and the user has searched using a term referring to a particular gene, then a visual indication such as an overlay is rendered over the map to indicate or in some way highlight the diseases that are associated with that gene. This highlighting creates a visual pattern that makes it easier for the user to visually recognise patterns in the results of which diseases are relevant—and to spot surprising characteristics of this pattern that may provide information for applications such as drug discovery that are not apparent when searching using traditional searching tools.

In a first aspect, the present disclosure provides a system for searching a set of biological entities, the system comprising: a user input module configured to receive a user input comprising a representation of a biological entity; a search module configured to determine which entities of a set of biological entities are associated with the user input; a visualisation module configured to render a visualisation of multiple biological entities of the set and of parent-child relationships between them; and an overlay module configured to render an association indicator visually indicating one or more biological entities of the visualisation that are associated with the user input.

Preferably, the set of biological entities comprises a set of diseases, genes, proteins, drugs, biological pathways, or biological processes.

Preferably, the user input comprises a representation of one or more of a disease, gene, protein, drug, biological pathway, biological process, anatomical region, anatomical entity, tissue, or cell type.

Preferably, the association indicator comprises an overlay.

Preferably, for each of the multiple biological entities, the visualisation comprises a visual indication of the respective biological entity, the visual indication having a size that depends on a hierarchical status of the respective biological entity in the parent-child relationships.

Preferably, the overlay module is configured to adapt a size of a visual indication of a biological entity based on an evidence type or confidence score of an association between the biological entity and the user input.

Preferably, the visualisation module is configured to render the visualisation by using a cartographic visualisation tool with non-spatial entities.

Preferably, the multiple biological entities comprise duplicated biological entities.

Preferably, the visualisation module is configured to enable zooming controlled by user input.

Preferably, the system is configured to enable user selection of the set of biological entities.

Preferably, the system is configured to render an entity-of-interest indicator visually indicating one or more biological entities having a threshold proportion of near relatives that are associated with the user input and are not themselves associated with the user input.

Preferably, the search module is configured to determine an association by querying a database.

Preferably, the database comprises association data curated by a user.

Preferably, the database comprises association data generated based on a machine learning prediction.

Preferably, the database comprises association data generated based on a co-occurrence in literature of the biological entity represented in the user input and a biological entity of the set of biological entities, the co-occurrence being detected by a natural language processing tool.

Preferably, the search module is configured to determine an association by causing a machine learning algorithm to generate a prediction.

Preferably, the search module is configured to determine an association by causing a natural language processing tool to detect at least one co-occurrence in literature of the biological entity represented in the user input and a biological entity of the set of biological entities.

Preferably, the overlay module is configured to render a visual indication of an evidence type of an association.

Preferably, the evidence type comprises human curation, machine learning prediction, or natural language processing.

Preferably, the evidence type comprises machine learning predication and the system comprises a filter module configured to enable the user to filter search results by setting a confidence score range of the machine learning prediction.

Preferably, the evidence type comprises natural language processing and the system comprises a filter module configured to enable the user to filter search results by setting a quantitative natural language processing evidence range.

Preferably, the system comprises a ring fencing module configured to enable a user to ring fence an area of the visualisation and to generate notifications when there are new associations or upgraded evidence types for associations in the ring-fenced area.

In a second aspect, the present disclosure provides a computer-implemented method of searching a set of biological entities, the method comprising: receiving a user input comprising a representation of a biological entity; determining which entities of a set of biological entities are associated with the user input; rendering a visualisation of multiple biological entities of the set and of parent-child relationships between them; and rendering an association indicator visually indicating one or more biological entities of the visualisation that are associated with the user input.

Preferably, the set of biological entities comprises a set of diseases, genes, proteins, drugs, biological pathways, or biological processes.

Preferably, the user input comprises a representation of one or more of a disease, gene, protein, drug, biological pathway, biological process, anatomical region, anatomical entity, tissue, or cell type.

Preferably, the association indicator comprises an overlay.

Preferably, for each of the multiple biological entities, the visualisation comprises a visual indication of the respective biological entity, the visual indication having a size that depends on a hierarchical status of the respective biological entity in the parent-child relationships.

Preferably, the method comprises adapting a size of a visual indication of a biological entity based on an evidence type or confidence score of an association between the biological entity and the user input.

Preferably, the method comprises rendering the visualisation by using a cartographic visualisation tool with non-spatial entities.

Preferably, the multiple biological entities comprise duplicated biological entities.

Preferably, the method comprises enabling zooming controlled by user input.

Preferably, the method comprises enabling user selection of the set of biological entities.

Preferably, the method comprises rendering an entity-of-interest indicator visually indicating one or more biological entities having a threshold proportion of near relatives that are associated with the user input and are not themselves associated with the user input.

Preferably, the method comprises determining an association by querying a database.

Preferably, the database comprises association data curated by a user.

Preferably, the database comprises association data generated based on a machine learning prediction.

Preferably, the database comprises association data generated based on a co-occurrence in literature of the biological entity represented in the user input and a biological entity of the set of biological entities, the co-occurrence being detected by a natural language processing tool.

Preferably, the method comprises determining an association by causing a machine learning algorithm to generate a prediction.

Preferably, the method comprises determining an association by causing a natural language processing tool to detect at least one co-occurrence in literature of the biological entity represented in the user input and a biological entity of the set of biological entities.

Preferably, the method comprises rendering a visual indication of an evidence type of an association.

Preferably, the evidence type comprises human curation, machine learning prediction, or natural language processing.

Preferably, the evidence type comprises machine learning predication and the system comprises a filter module configured to enable the user to filter search results by setting a confidence score range of the machine learning prediction.

Preferably, the evidence type comprises natural language processing and the system comprises a filter module configured to enable the user to filter search results by setting a quantitative natural language processing evidence range.

Preferably, the method comprises enabling a user to ring fence an area of the visualisation and to generate notifications when there are new associations or upgraded evidence types for associations in the ring-fenced area.

In a third aspect, the present disclosure provides a system for searching a set of entities, the system comprising: a user input module configured to receive a user input comprising a representation of an entity; a search module configured to determine which entities of a set of entities are associated with the user input; a visualisation module configured to render a visualisation of multiple entities of the set and of parent-child relationships between them; and an overlay module configured to render an association indicator visually indicating one or more entities of the visualisation that are associated with the user input.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a schematic diagram of a module view of a system for searching a set of entities according to the present disclosure;

FIG. 2 is a block diagram of hardware suitable for implementing a system for searching a set of entities according to the present disclosure;

FIG. 3 is a flow chart showing a method of searching a set of entities according to the present disclosure;

FIG. 4 is a screenshot showing a portion of a two-dimensional visualisation of a set of diseases;

FIG. 5 is a schematic diagram showing hierarchical relationships between a small subset of diseases including a disease having two parent diseases;

FIG. 6 is a screenshot showing a portion of a two-dimensional visualisation of a set of diseases in which a disease and its two parent diseases are emphasised;

FIG. 7 is a screenshot of the whole visualisation of FIG. 6 showing its hair-ball structure;

FIG. 8 is a FIG. 5 is a schematic diagram showing hierarchical relationships between a small subset of diseases with duplication of entities;

FIG. 9 is a screenshot showing a portion of a two-dimensional visualisation of a set of diseases in which a disease having two parent diseases is duplicated;

FIG. 10 is a screenshot of the whole visualisation of FIG. 9 showing its clustered structure;

FIG. 11 is a screenshot of diseases associated with a particular gene overlaid on a visualisation of a set of all diseases;

FIG. 12 is a screenshot of diseases associated with a particular disease overlaid on a visualisation of a set of all diseases;

FIG. 13 is a schematic diagram of diseases associated with a particular gene overlaid on a visualisation of a set of all diseases;

FIG. 14 is a schematic diagram indicating an odd-one-out disease surrounded by diseases that are associated with a particular gene;

FIG. 15 is a schematic diagram of indicating diseases proximal to diseases that are associated with a particular gene;

FIG. 16 is a schematic diagram of example associations between biological entities;

FIG. 17 is a schematic diagram showing visual indications of three suitable types of evidence for associations; and

FIG. 18 is a screenshot showing a search result filter panel.

Common reference numerals are used throughout the figures to indicate similar features.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

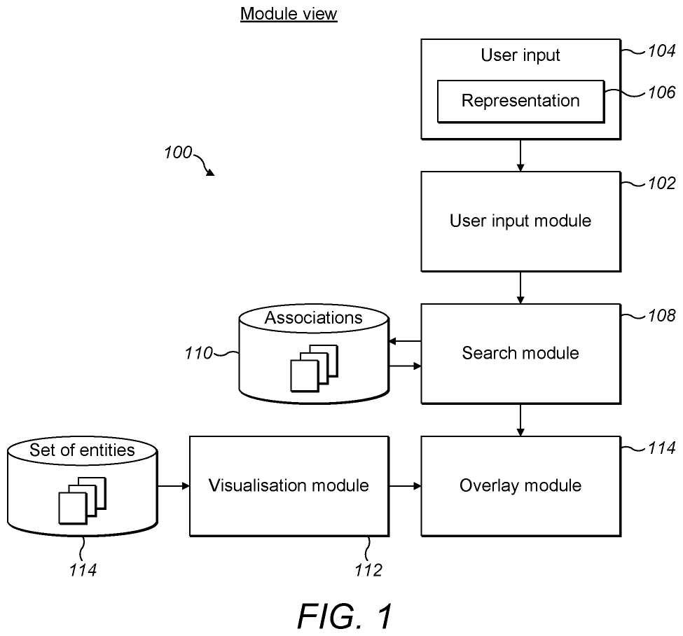

FIG. 1 illustrates a module view of a system 100 for searching a set of entities according to the present disclosure. The system 100 includes a user input module 102 configured to receive a user input 104 comprising a representation 106 of an entity. The represented entity can be thought of as a searching entity provided by the user for the purpose of searching the set of entities. For example, in the drug discovery field, a user may wish to search for which diseases in the set of all diseases are associated with a particular gene. In this case, the set of entities is the set of diseases and the user input 104 comprises a representation 106 of the gene. In a variation, the user input 104 may comprise a representation of multiple entities, such as a representation of two genes, or a representation of a gene and a drug. In this case, if searching the set of diseases, the search is for diseases that are associated with the two genes, or with the gene and the drug. Thus, the representation of multiple entities may comprise a series or list of representations of individual entities. Alternatively the multiple entities may comprise a prefix followed by a wildcard, for example to denote a group of related genes. In this case the search would be for diseases that are related to all the genes in the group.

The system 100 comprises a search module 108 communicatively connected to the user input module 104 such that the user input module 102 may provide information from the user input 104, such as the representation 106 of the searching entity, to the search module 108. The search module 108 is configured to determine which entities of the set of entities are associated with the user input 104. This may be implemented by way of the search module 108 interrogating a database. For example, the search module 108 may be communicatively connected to an associations database 110 which may be comprised as part of the system 100, or alternatively may be external to the system 100. The associations database 110 may store information relating to known associations between entities of various types. For example, in the drug discovery field, the associations database 110 may store information relating to known associations between diseases and other diseases, known associations between diseases and genes, or known associations between diseases and biological pathways. By interrogating the associations database 110, the search module 108 is able to establish which diseases are associated with a particular gene, or which diseases are associated with a particular disease, and so on, according to the content of the user input 104. It will be appreciated that a biological pathway may be defined as a sequence of events between a set of genes that can cause or prevent a biological process, such as cell death. Typically, a combination of processes and pathways are described in the context of a disease as ‘mechanisms’ which are of interest when wanting to prevent, treat or cure a disease.

The system 100 also includes a visualisation module 112 which is communicatively connected to an entities database 114. The entities database 114 stores a set of entities and their inter-relationships, and may be part of the system 100 or may be external to the system 100. The visualisation module 112 is configured to render a visualisation of the set of entities and of a set of parent-child relationships between them. The visualisation comprises a visual indication of each entity of the set, each entity being related to at least one other entity of the set by a parent-child relationship. This provides a visual representation of the whole set of entities that is based on the hierarchical relationships, such as child-parent and child-grandparent relationships, existing between the entities.

The system 100 also includes an overlay module 114 communicatively connected to the search module 108 and the visualisation module 112. The overlay module 114 is configured to render an overlay over the visualisation indicating which entities are associated with the user input 104. As a result, the system 100 is configured to render a visualisation of the set of entities and then to overlay on top of this an indication of which entities of the set are associated with the user input 104. For example, in the drug discovery field, if a user wants to search for diseases that are associated with a particular gene, then the system 100 can render a visualisation of all diseases and overlay

on top of that an indication of which diseases are associated with the gene. This enables the user to view the diseases associated with the gene that have come up in the search in the context of the full set of diseases.

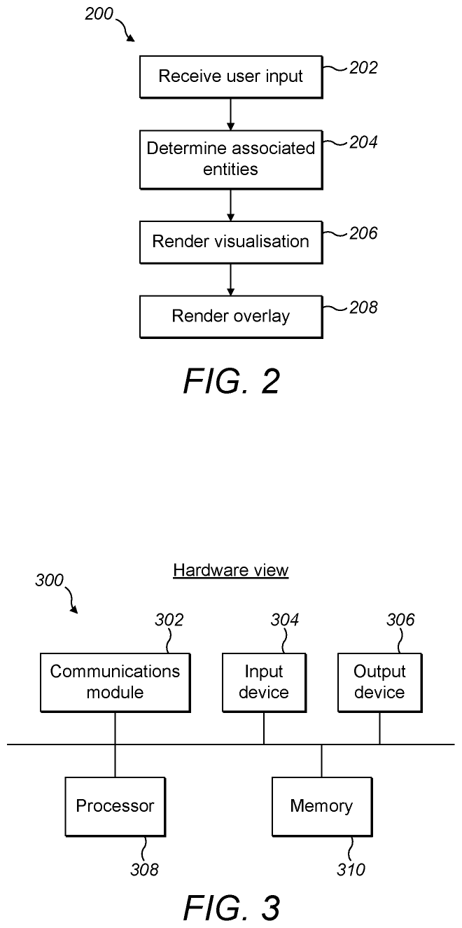

With reference to FIG. 2, the present disclosure includes a computer-implemented method 200 of searching a set of entities, the method 200 comprising: receiving 202 a user input comprising a representation of an entity; determining 204 which entities of a set of entities are associated with the user input; rendering 206 a visualisation of the set of entities and their inter-relationships, the visualisation comprising one or more clusters of the entities in which each entity of a respective cluster is related to at least one other entity of the respective cluster by a parent-child relationship; and rendering 208 an overlay over the visualisation indicating which entities are associated with the user input.

Referring to FIG. 3, the method 200 may be implemented using hardware 300. The hardware 300 includes a communications module 302, an input device 304 suitable for receiving a user input, an output device 306 which may comprise a display, a processor 308, and memory 310 which may suitably store a program that when run causes the processor to implement the method 200.

Hierarchical relationships between entities of a set are relationships between entities of the set in which one entity has a higher hierarchical status than the other. For example, a hierarchical disease ontology or classification system provides a hierarchical catalogue, that may be manually curated, of all diseases in which each disease is related to another in a parent-child relationship. Generally, the parent disease is a broader term and the child disease is a narrower term. For example, a parent-child relationship may exist between a broader parent disease ‘eye disease’ and a narrower child disease ‘retinal disease’. In this document, the term ‘disease’ includes specific diseases as well as classes of diseases such as the class of eye diseases. Other hierarchical relationships such as grandparent-child relationships and sibling relationships may be inferred from multiple child-parent relationships.

Any set of entities having hierarchical inter-relationships that include parent-child relationships can be searched using the system 100 or method 200. For example, in the biological space the set of entities may comprise a set of biological entities such as diseases, genes, proteins, drugs, biological pathways, biological processes, anatomical regions or entities, tissues, or cell types. In this case the user input may suitably comprise a representation of a biological entity, for example a disease, gene, protein, drug, biological pathway, biological process, anatomical regions or entities, tissues, or cell types. In the biological space, the set of entities may alternatively comprise a set of entities that are related to a biological entity. For example, the set of entities may comprise a set of patents or a set of clinical trials that are related to a disease or a class of diseases. In other fields, the set of entities may comprise a set of entities such as sports, family members, pipes in a sewers network, Wikipedia pages, documents in a library, and published patents.

By way of example, details of the present disclosure will now be described by way of reference to biological entities. As such, it will be appreciated that the present disclosure includes a system for searching a set of biological entities, the system comprising: a user input module configured to receive a user input comprising a representation of a biological entity; a search module configured to determine which entities of a set of biological entities are associated with the user input; a visualisation module configured to render a visualisation of the set of biological entities and of a set of parent-child relationships between them, the visualisation comprising a visual indication of each biological entity of the set, each biological entity being related to at least one other biological entity of the set by a parent-child relationship; and an overlay module configured to render an overlay over the visualisation indicating which biological entities are associated with the user input.

The present disclosure also includes a computer-implemented method of searching a set of biological entities, the method comprising: receiving a user input comprising a representation of a biological entity; determining which entities of a set of biological entities are associated with the user input; rendering a visualisation of the set of biological entities, the visualisation comprising one or more clusters of the biological entities in which each biological entity of a respective cluster is related to at least one other biological entity of the respective cluster by a parent-child relationship; and rendering an overlay over the visualisation indicating which biological entities are associated with the user input.

In particular, the details of the present disclosure will be described by way of reference to a visualisation of a set of diseases. In the example provided, the system 100 is configured to render a visualisation of a comprehensive set of diseases, containing around 20,000 diseases. This is therefore a visualisation of a very large set of information, showing all diseases visually in a map-like display to the user, which is useful for assisting the user in browsing areas of the visualisation, and in forming mental models of the full set of diseases and the relationships between them.

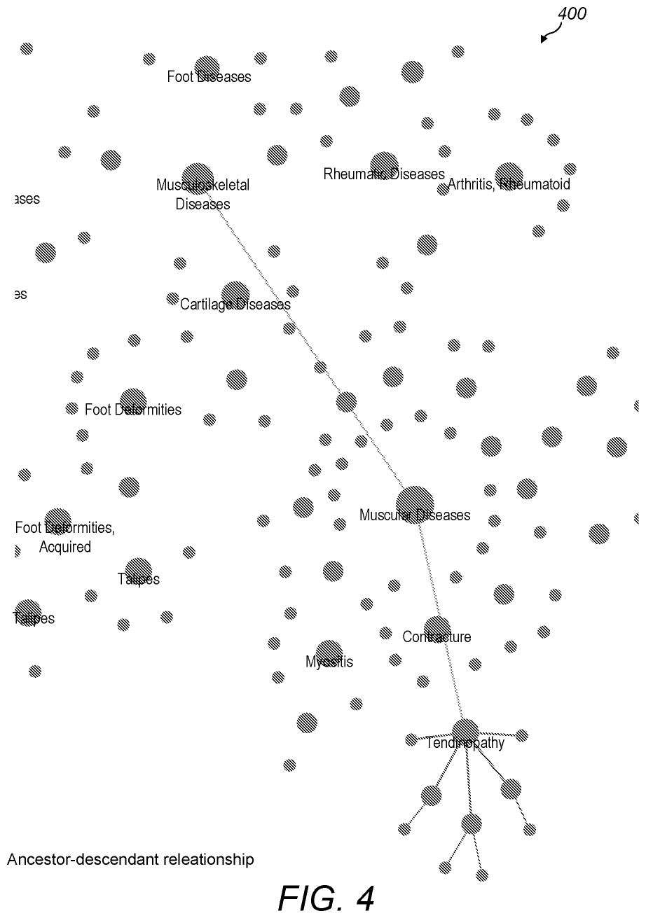

FIG. 4 shows a portion 400 of a two-dimensional visualisation of a set of diseases. Each disease is represented by a visual indication of a disease, in this case in the form of a filled circle. Some of the diseases, such as musculoskeletal diseases, cartilage diseases and foot diseases, are labelled with their names in accordance with the zoom level. As shown in FIG. 4, at a given zoom level the visual indications of the diseases may vary in size in dependence on the relative levels of the diseases in the hierarchy. For example, muscular diseases has a larger filled circle than myositis and contracture because myositis and contracture are child diseases of muscular diseases.

The visualisation includes visual indications of parent-child relationships between the diseases. As shown in FIG. 4, these may be provided in the form of straight lines connecting the parent and child diseases. For example, a line connects myositis to its parent, muscular diseases. Similarly, five further lines connect myositis to its five child diseases. Visual representations of child diseases may be fanned out from their parents to fill the space using a range of techniques such as, for example, using a spring algorithm.

The visualisation module may be configured to render the visualisation by using a cartographic visualisation tool with non-spatial entities. A cartographic visualisation tool is intended to be used with spatial entities such as geographical or spatial coordinates of some kind, such as longitude and latitude coordinates. Cartographic visualisation tools have been developed over many years to deal with geographic and urban complexity, from terrains and gradients to roads and walkway labels. The technology can be repurposed to visualise non-spatial data, thereby benefiting users in non-spatial applications in terms of high performance and smooth interaction. To achieve this, non-spatial data is transformed to spatial data. For example, geometric shapes such as lines and polygons used to show a graph of relationships between entities may be converted to spatial data, such as those found in the GeoJSON specification.

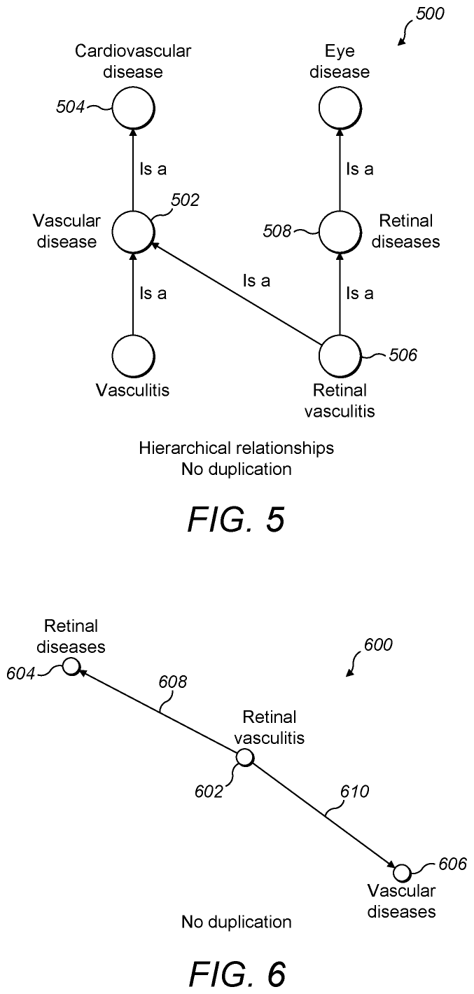

FIG. 5 shows a structure 500 of hierarchical relationships between a small subset of diseases. Each child-parent relationship is indicated by an arrow connecting a child disease to a parent disease. For example, it can be seen that vascular disease 502 is a child disease of cardiovascular disease 504. Some diseases have multiple parent diseases, and an example of this is retinal vasculitis 506 in FIG. 5 which has two parent diseases: vascular disease 502 and retinal disease 508. This comes about because retinal vasculitis 506 is both a vascular disease 502 and a retinal disease 508.

In a visualisation of the set of diseases, a disease having two parent diseases may be placed between its parents. For example, FIG. 6 shows a portion 600 of a visualisation of a set of diseases in which retinal vasculitis 602 is placed between its two parents, retinal disease 604 and vascular disease 606. The two child-parent relationships are indicated visually by an arrow 608 from retinal vasculitis 602 to its parent retinal disease 604 and an arrow 610 from retinal vasculitis 602 to its other parent vascular disease 606.

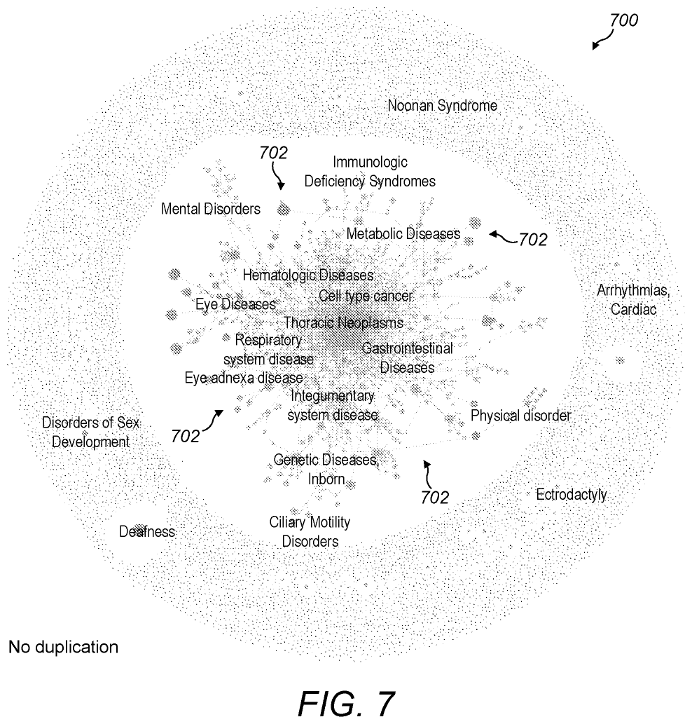

As shown in FIG. 7, when one zooms out from the portion 600, the whole of the visualisation 700 comes into view. The visualisation 700 places most of the diseases in a central hair-ball structure which is difficult to navigate. This is a result of the size of the set of entities (there are around 20,000 diseases in total) and the complexity of their inter-relationships. Since diseases are a complex biological set of entities, there are complex inter-linkages between them, for example with many diseases having multiple parents, creating complex links between diseases in different categories. Some diseases have no classification due to rarity or specificity, resulting in a disconnection from the rest of the hierarchical structure. The layout algorithm uniformly distributes unconnected diseases around the central hierarchical hair-ball structure in a ring-like shape to retain them in the same view.

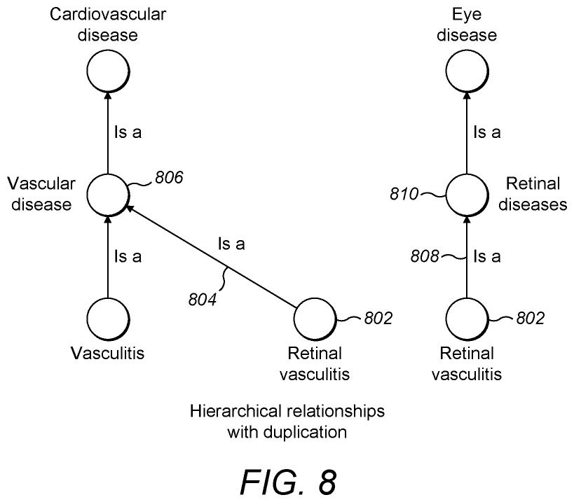

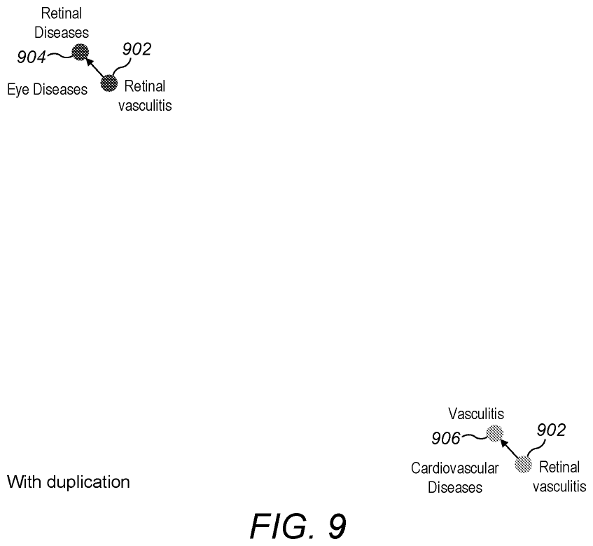

For large and complex sets of entities such as diseases, it is suitable to simplify the tangled visualisation by duplicating entities. For example, referring to FIG. 8, a disease such as retinal vasculitis having two parent diseases may be duplicated to appear twice. In this case, retinal vasculitis 802 appears twice, once with an arrow 804 representing its relationship with its parent vascular disease 806, and once with an arrow 808 representing its relationship with its parent retinal disease 810.

Based on this approach, a visualisation of the set of diseases may show retinal vasculitis twice, once in the region of its parent retinal diseases and once in the region of its parent vasculitis. As shown in FIG. 9, retinal vasculitis 902 appears with its parent retinal diseases 904 in an area of eye diseases, and retinal vasculitis 902 appears again with its parent vasculitis 906 in a region of cardiovascular diseases.

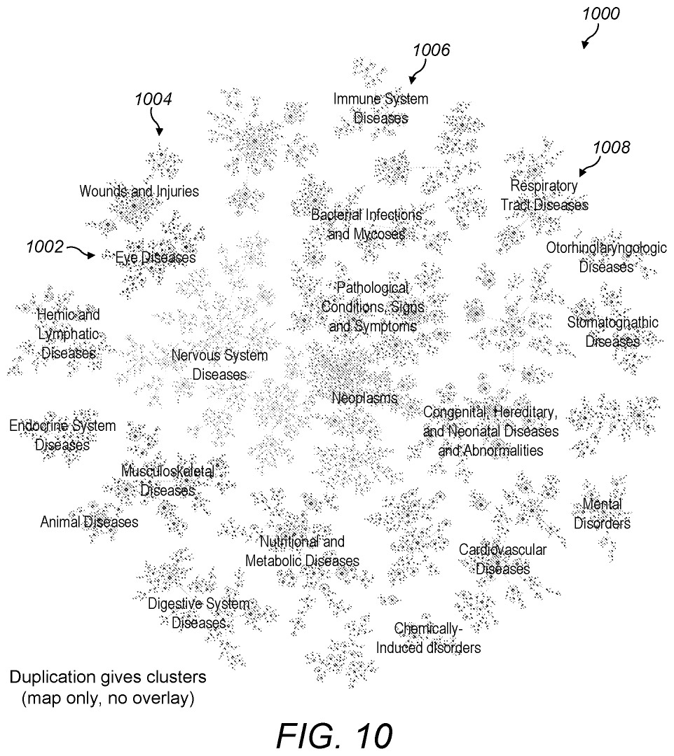

These regions may be referred to as clusters since the set of all diseases naturally separates out into 27 clusters when the approach of duplicating entities with multiple parents is followed. As shown in FIG. 10, the whole visualisation 1000 with duplicated diseases includes clusters such as eye diseases 1002, wounds and injuries 1004, immune system diseases 1006, and respiratory tract diseases 1008.

The visualisation 1000 with duplicated diseases may be viewed at different zoom levels. For example, a fairly zoomed out zoom level may place the set of diseases zoomed out to the point where the whole set is shown in a small area. At this zoom level, it may be suitable for only some of the clusters to be labelled. Clusters may be labelled with the name of the disease that is highest in the hierarchy of relationships in that cluster.

A slightly more zoomed in zoom level may show all the names of the clusters and some more detail of each cluster. It may be convenient to show each cluster in a unique colour to help differentiate them visually, particularly at the lower zoom levels where the view is not very zoomed in.

Further zoomed in zoom levels may show the cluster names and the details of the clusters in further detail.

At a sufficiently zoomed in level, names of diseases within each cluster may be introduced. As the rendering becomes progressively zoomed in, lower levels in the hierarchy of diseases become less crowded and can be more easily labelled. Diseases in lower levels of the hierarchy of relationships are nested around their parents, for example being spatially distributed by a spring algorithm. Diseases in lower levels may also be represented by a visual indication such as a filled circle that are smaller than the visual indications of their parents. This provides a clear signal to the viewer of the relative status in the hierarchy of relationships of the various child and parent diseases. The user can zoom to the higher zoom levels (i.e. zoom in) to make lower diseases in the hierarchy the current viewing level.

In the example we have seen in FIGS. 4-10, the biological entities shown in the visualisation are diseases, but this does not always have to be the case. As indicated above, the set of biological diseases may comprise for example a set of diseases, genes, proteins, drugs, biological pathways, or biological processes. In some implementations, the system may enable the user to select a set of biological entities that are to be visualised by the visualisation module. This enables the user to use the system to search for which biological entities of a user-selected set are associated with a user-selected biological entity. For example, in a first search the user could be looking for diseases associated with a particular gene, and in a second search the user could be looking for biological pathways associated with a particular drug. For the first search the visualisation module generates a visualisation of the set of diseases, while for the second search the visualisation module generates a visualisation of the set of biological pathways.

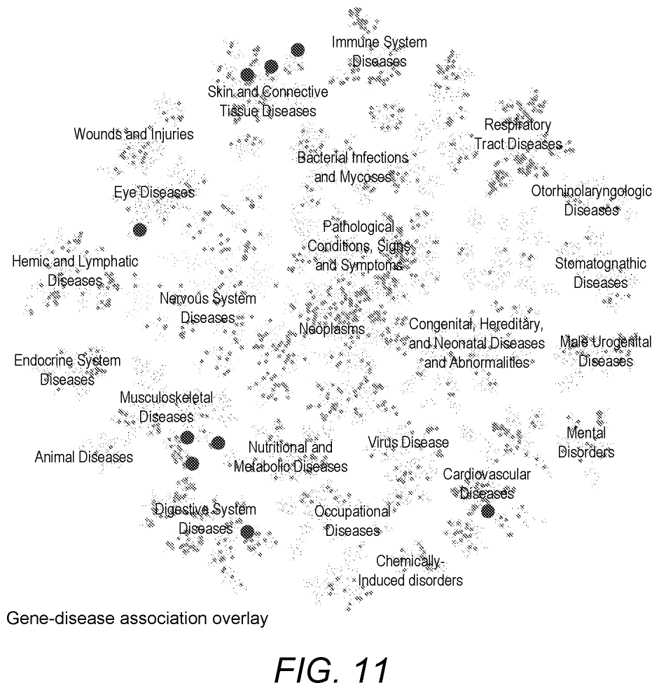

As indicated above, a system of the present disclosure includes an overlay module configured to render an overlay over the visualisation indicating which biological entities are associated with a user input. For example, if a user wishes to search for diseases associated with a particular gene, the system may be configured to render, on top of a visualisation of the set of all diseases, an overlay indicating which of the diseases is associated with the gene. An example of this is shown in FIG. 11 where a visualisation of the set of all diseases is rendered with an overlay of diseases showing up in the search as being associated with the gene. Only way of implementing the overlay comprises simply de-emphasising the diseases that are not part of the search results by reducing their colour density. Alternatively, the colour density of the diseases to be overlaid could be increased. Various other ways, such as using highlighting colours or other visual indications could be used to implement the overlay.

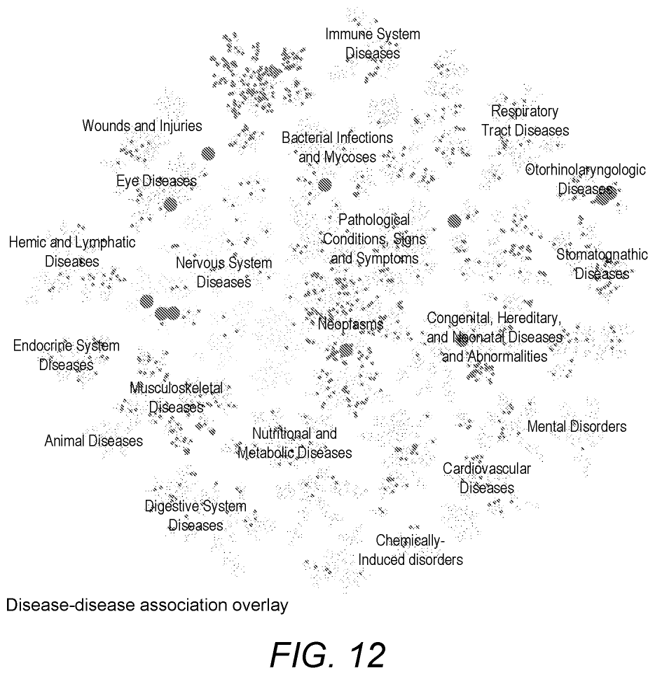

Another example of an overlay over a visualisation of the set of all diseases is shown in FIG. 12. Here, a search has been done to find diseases associated with a particular disease. Those that are found to be relevant are emphasised by rendering an overlay over the visualisation. In the example shown in FIG. 12, the overlay is implemented by de-emphasising (with a reduced colour density) the diseases found not to be associated with the particular disease.

As it can be appreciated from the overlays shown in FIGS. 20 and 21, the overlays present visual patterns of search results to the user. These visual patterns may, for example, comprise spatial clustering of search results. Clusters of various sizes may provide a drug discoverer user reviewing the overlay with various hints and clues as to potentially new discoveries in the drug discovery and wider biological fields.

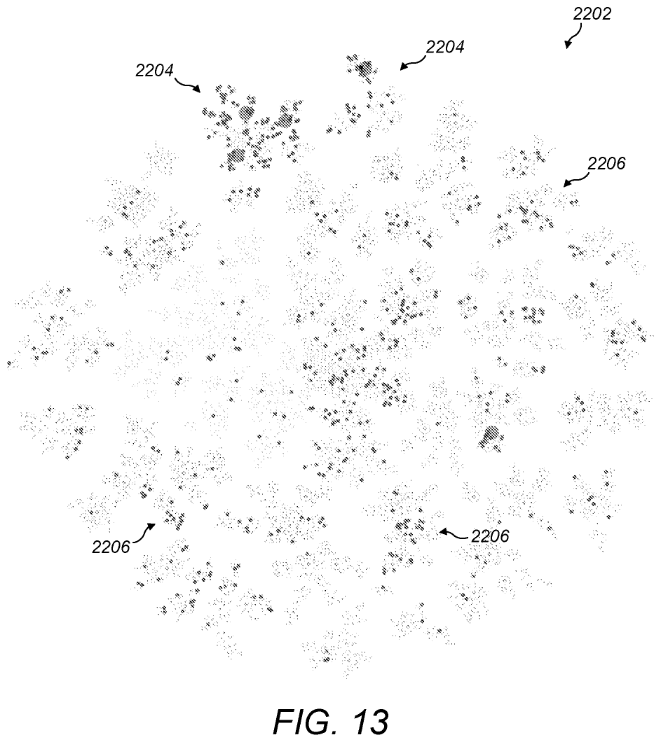

For example, with reference to FIG. 13, a search for diseases associated with a particular gene may result in an overlay over a visualisation 2202 of the set of all diseases, the overlay comprising expected clusters 2204 and an unexpected cluster 2206. The spatial proximity of the diseases in the unexpected cluster 2206 makes these search results easy to spot. Furthermore, the combination of the unexpected cluster 2206 with the expected clusters 2204 may indicate that the diseases of the unexpected cluster 2206 could have the same mechanism as, and be treatable by the same drugs as, the diseases of the expected clusters 2204.

In general, spatial clustering of related results makes them easy to spot, helping to resolve the information overload problem. Diseases that are related to each other, and might be identified in a traditional list of search results on pages 100, 204 and 506, will show up in a small cluster. Not only does the clustering make the small group easier to see, but the spatial proximity of these diseases emphasises to the user that the diseases are related. Showing up together in a small cluster like this may provide a hint that the diseases of the small cluster have a common mechanism, and therefore may respond to the same drugs.

At time same time, if diseases in a few areas of a visualisation of the set of all diseases show up strongly in a gene search, this could give also a clue that they may have the same mechanism and this may also give a drug discoverer a clue as to what that mechanism might be.

By overlaying the associations between a gene and a set of diseases, hidden relationships such as potential disease mechanisms can be surfaced through the visual patterns appearing in the overlay. This approach of overlaying search results over a visualisation of a set of entities has the advantage of visually surfacing hidden relationships between search results through spatial patterns that emerge in the overlay. This cannot be achieved using the traditional approach of presenting search results to a user in an ordered list.

Finally, it was indicated above that for large sets of entities, such as the set of around 20,000 diseases, it is suitable to duplicate entities in the visualisation to avoid the hair ball effect and instead create well differentiated clusters of entities in the visualisation. In this case, a duplicated entity showing up as a search result will be highlighted in multiple locations as part of the overlay. This may prompt a user to consider different areas of the visualisation. For example, a drug discoverer may be prompted to start thinking about using a drug in a non-traditional family of diseases if the overlay presents a disease associated with the drug in multiple areas of the visualisation.

It can be appreciated that displaying search results as an overlay in the ways described above is associated with several advantages. However, there are also advantages flowing from the spatial patterns arising in the entities near the search results. For example, if a search is conducted for diseases relevant to a particular gene, then diseases that are near the search results but are not search results themselves may provide useful information.

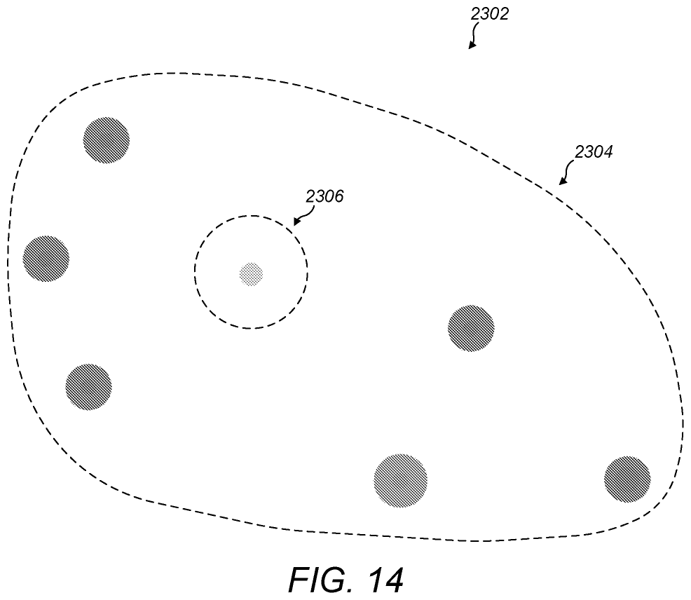

For example, referring to FIG. 14, a visualisation 2302 of the set of all diseases may be rendered. If the user has searched for diseases associated with a particular gene, then the associated diseases that show up as search results are indicated by rendering an overlay. The overlay may comprise clusters 2304 of diseases that are found to be associated with the gene. One of the clusters 2304 may include a group of diseases that are close family members (e.g. parent, child, sibling and grandparent diseases) in close proximity to a near relative 2306 that has not shown up as a search result. The near relative becomes conspicuous because it can be seen as part of the rendered visualisation of the set of all diseases, but it is near to, or even surrounded by, several close family members that have all shown up as search results in the overlay. This makes the odd-one-out 2306 easy to spot. Such odd-one-out diseases may present interesting new possibilities for targeted research. An odd-one-out disease could, for example, respond to similar drugs to its family members in a way that has not previously been discovered. This approach is a significant advantage over traditional ordered list presentation of search results because the odd-one out would not even appear in the list at all in the traditional approach.

To make it even easier to identify odd-one-out type entities, the system may be configured to render a visual indication of each biological entity of the visualisation that has a threshold proportion or number of near relatives in the overlay and is not itself included in the overlay. This visual indication of odd-one-out entities may, for example, be implemented using a reserved colour, a symbol, or a ring rendered around such entities. Near relatives are diseases having a threshold similarity to each other. The similarity metric may be based on one or more similarity measures such as similarity of disease classification, similarity of disease mechanism, or similarity of disease anatomy.

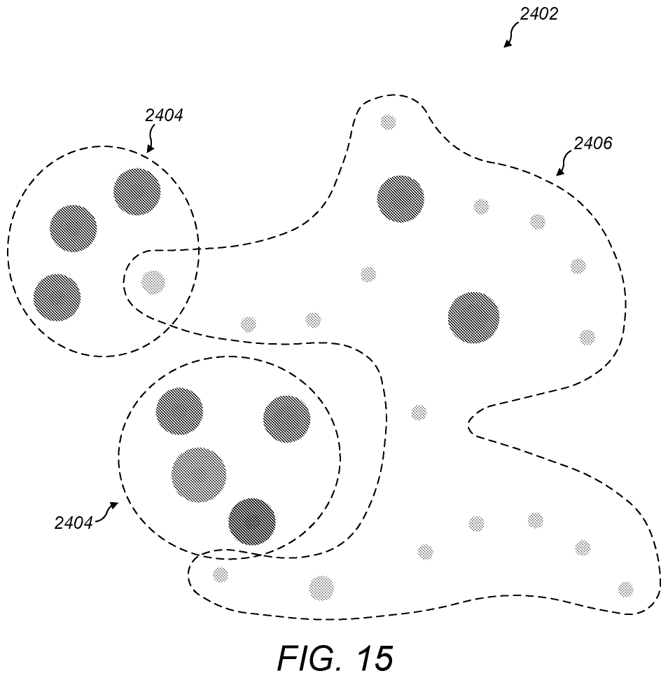

Similarly, near relatives that are not necessarily odd-one-out diseases, but are simply near to a cluster of diseases in an overlay, may also provide an opportunity for research. As shown in FIG. 15, if a user searches for diseases associated with a particular gene, a visualisation 2402 of the set of all diseases may be rendered and clusters 2404 of associated diseases may be overlaid. There may be a group of diseases in the visualisation 2402 but not appearing in the overlay that are proximal to one of the clusters 2404. The proximal diseases 2406 are easy to spot by a user because they show up visually next to a cluster 2404 of the overlay. In some implementations, the system may be configured to render a visual indication of each entity of the visualisation that has a threshold proximity to entities of the overlay and is not itself included in the overlay. This supports the user my making it even easier to see which are the proximal diseases that may be worth investigating in targeted research. In this context, proximal entities may be defined as entities that are within a threshold number of “hops” (i.e. parent-child relationships) from each other. For example, if proximal entities are defined as being up to two hops away from each other, then parent and child diseases are proximal to each other, grandparent and child diseases are proximal to each other, and sibling diseases are proximal to each other.

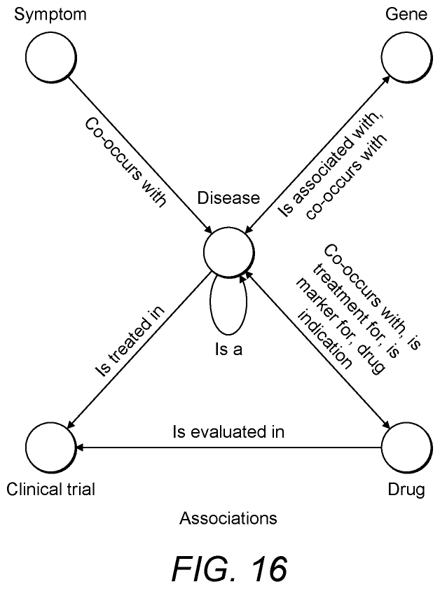

There are various types of associations that can exist between biological entities. For example, an association between a disease and a gene could mean that the disease co-occurs with the gene. Similarly, an association between a disease and a drug could mean that the disease co-occurs with, is treatment for, or is a marker for the drug. FIG. 16 shows example associations between five types of biological entities: diseases, genes, drugs, symptoms, and clinical trials. It is also possible for a biological entity to have a relationship with another biological entity of the same type, for example a disease may be a sub-category of another disease (e.g. retinal vasculitis is a retinal disease).

The search module may be configured to determine associations in various ways. For example, some associations can be established based on human curation. This may be implemented by a scientific curator manually annotating the association in a database, and is considered to be very reliable. An association that is curated may be considered a fact.

Another evidence type is prediction using a machine learning algorithm that extracts associations from literature. The algorithm may be configured to assign a confidence score between 0 (no confidence) and 1 (total confidence). Machine learning prediction with high scores may be considered to provide strong evidence for an association. Literature ingested as source information may include sources such as scientific journals, biomedical databases, patents, and so on.

Co-occurrence in literature, for example co-occurrence in the same sentence in literature, detected by natural language processing (NLP), offers another evidence type. Co-occurrence is considered to be weak evidence because the meaning of the sentence is not taken into account. However, a confidence score may still be assigned, for example based on the number of articles in which a co-occurrence is found. Literature parsed as source information may include sources such as scientific journals, patents, and so on.

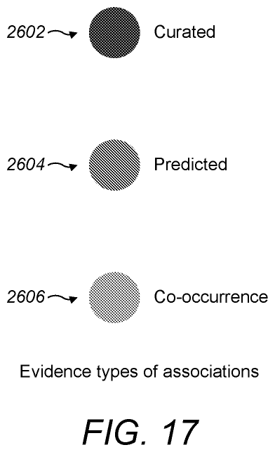

The overlay module may be configured to render an overlay comprising a visual indication (such as colour coding) of an evidence type. For example, referring to FIG. 17, entities found to be associated with a user input based on curated evidence may be represented by a green indication 2602 in the overlay. Similarly, entities with associations based on machine learning prediction may be represented by a red indication 2604, and entities with associations based on NLP evidence may be represented by a blue indication 2606. Other colours or visual indications may also be suitable. Rendering a visual indication of the type of evidence builds user trust in the system and helps to convey how reliable the evidence for the association is.

Confidence scores for associations based on machine learning or NLP may also be visually indicated in the overlay. For example, the size of a visual indication of an entity may be increased for higher confidence scores and reduced for lower confidence scores. It may be suitable to set limits on the range of sizes available for different confidence scores to ensure that parent diseases are still generally larger than their children. The size adaptation based on confidence scores may also help to build user trust in the system as it is conveyed how reliable a particular machine learning prediction is considered to be or how frequent the co-occurrence in the literature is.

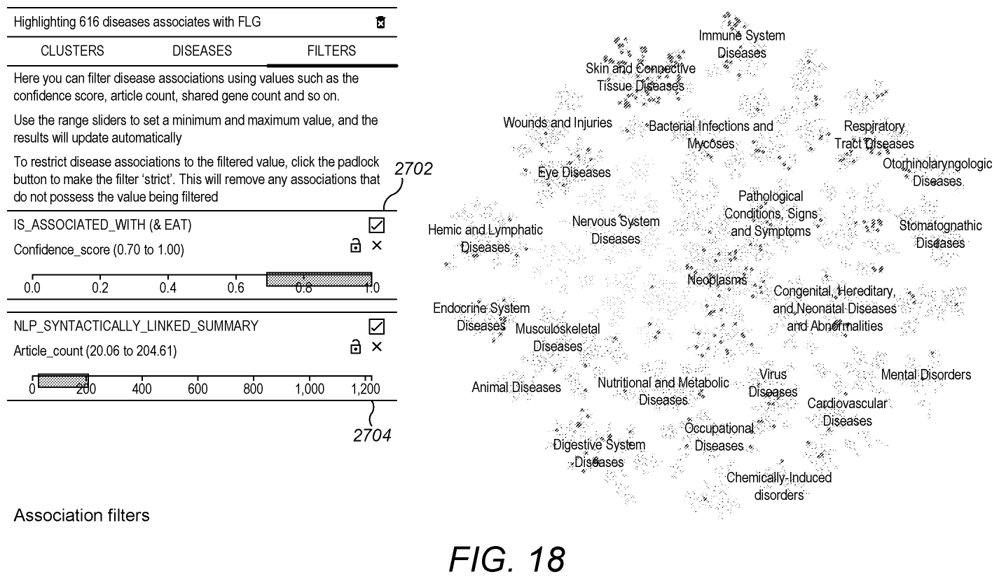

Confidence scores for machine learning predictions or NLP-based evidence may also be used for filtering search results. For example, referring to FIG. 18, a user may want to only include search results based on machine learning if they have confidence scores between 0.7 and 1.0. This can be selected in a filter window 2702. Similarly, using another filter window 2704, a user may want to only include search results based on NLP evidence if co-occurrence is detected in up to, say, 200 articles or 1000 sentences. This may assist in looking for patterns or relationships between diseases and a gene that are predicted by machine learning with high confidence but may be little known in the literature. A range of quantitative NLP evidence, such as a range of how many articles or sentences in which co-occurrence is to be detected, may be specified by the user to filter the results. The range may include a minimum number of articles or sentences in which co-occurrence is preferred by the user to be detected. Controlling confidence scores and quantitative NLP evidence ranges in this way to filter results may therefore assist the user in discovering unknown relationships. This type of control may also help to reduce the user's experience of information overload, and may assist in helping the user to trust the system and to exert some control over the search results.

As new scientific research results are generated in the scientific community, new scientific articles and other information sources are created. These can be used to update machine learning based and NLP based associations. At the same time, further human curation of associations may be added to a database. With this in mind, the system may include a ring fencing module configured to enable a user to ring fence an area of a visualisation of a set of biological entities and to generate notifications when there are new associations or upgraded evidence types for associations in the ring-fenced area. This may assist a user if they are particularly interested in an area of a visualisation, for example a particular subset of diseases, and want to keep track of any developments.

In the embodiment described above the server may comprise a single server or network of servers. In some examples, the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic. In some examples, a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and Blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fibre optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence.

For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A system for searching a set of biological entities, the system comprising:
a user input module configured to receive a user input comprising a representation of a biological entity;
a search module configured to determine which entities of a set of biological entities are associated with the user input;
a visualisation module configured to render a visualisation of multiple biological entities of the set and of parent-child relationships between them; and
an overlay module configured to render an association indicator visually indicating one or more biological entities of the visualisation that are associated with the user input.

2. A system according to claim 1, wherein the set of biological entities comprises a set of diseases, genes, proteins, drugs, biological pathways, or biological processes.

3. A system according to claim 1, wherein the user input comprises a representation of one or more of a disease, gene, protein, drug, biological pathway, biological process, anatomical region, anatomical entity, tissue, or cell type.

4. A system according to claim 1, wherein the association indicator comprises an overlay.

5. A system according to claim 1, wherein, for each of the multiple biological entities, the visualisation comprises a visual indication of the respective biological entity, the visual indication having a size that depends on a hierarchical status of the respective biological entity in the parent-child relationships.

6. A system according to claim 1, wherein the overlay module is configured to adapt a size of a visual indication of a biological entity based on an evidence type or confidence score of an association between the biological entity and the user input.

7. A system according to claim 1, wherein the visualisation module is configured to render the visualisation by using a cartographic visualisation tool with non-spatial entities.

8. A system according to claim 1, wherein the multiple biological entities comprise duplicated biological entities.

9. A system according to claim 1, wherein the visualisation module is configured to enable zooming controlled by user input.

10. A system according to claim 1, wherein the system is configured to enable user selection of the set of biological entities.

11. A system according to claim 1, wherein the system is configured to render an entity-of-interest indicator visually indicating one or more biological entities having a threshold proportion of near relatives that are associated with the user input and are not themselves associated with the user input.

12. A system according to claim 1, wherein the search module is configured to determine an association by querying a database.

13. A system according to claim 12, wherein the database comprises association data curated by a user.

14. A system according to claim 12, wherein the database comprises association data generated based on a machine learning prediction.

15. A system according to claim 12, wherein the database comprises association data generated based on a co-occurrence in literature of the biological entity represented in the user input and a biological entity of the set of biological entities, the co-occurrence being detected by a natural language processing tool.

16. A system according to claim 1, wherein the search module is configured to determine an association by causing a machine learning algorithm to generate a prediction.

17. A system according to claim 1, wherein the search module is configured to determine an association by causing a natural language processing tool to detect at least one co-occurrence in literature of the biological entity represented in the user input and a biological entity of the set of biological entities.

18. A system according to claim 1, wherein the overlay module is configured to render a visual indication of an evidence type of an association.

19. A system according to claim 18, wherein the evidence type comprises human curation, machine learning prediction, or natural language processing.

20. A system according to claim 19, wherein the evidence type comprises machine learning predication and the system comprises a filter module configured to enable the user to filter search results by setting a confidence score range of the machine learning prediction.

21. A system according to claim 19, wherein the evidence type comprises natural language processing and the system comprises a filter module configured to enable the user to filter search results by setting a quantitative natural language processing evidence range.

22. A system according to claim 1, comprising a ring fencing module configured to enable a user to ring fence an area of the visualisation and to generate notifications when there are new associations or upgraded evidence types for associations in the ring-fenced area.

23. A computer-implemented method of searching a set of biological entities, the method comprising:
receiving a user input comprising a representation of a biological entity;
determining which entities of a set of biological entities are associated with the user input;
rendering a visualisation of multiple biological entities of the set and of parent-child relationships between them; and
rendering an association indicator visually indicating one or more biological entities of the visualisation that are associated with the user input.

24. A method according to claim 23, wherein the set of biological entities comprises a set of diseases, genes, proteins, drugs, biological pathways, or biological processes.

25. A method according to claim 23, wherein the user input comprises a representation of one or more of a disease, gene, protein, drug, biological pathway, biological process, anatomical region, anatomical entity, tissue, or cell type.

26. A method according to claim 23, wherein the association indicator comprises an overlay.

27. A method according to claim 23, wherein, for each of the multiple biological entities, the visualisation comprises a visual indication of the respective biological entity, the visual indication having a size that depends on a hierarchical status of the respective biological entity in the parent-child relationships.

28. A method according to claim 23, comprising adapting a size of a visual indication of a biological entity based on an evidence type or confidence score of an association between the biological entity and the user input.

29. A method according to claim 23, comprising rendering the visualisation by using a cartographic visualisation tool with non-spatial entities.

30. A method according to claim 23, wherein the multiple biological entities comprise duplicated biological entities.

31. A method according to claim 23, comprising enabling zooming controlled by user input.

32. A method according to claim 23, comprising enabling user selection of the set of biological entities.

33. A method according to claim 23, comprising rendering an entity-of-interest indicator visually indicating one or more biological entities having a threshold proportion of near relatives that are associated with the user input and are not themselves associated with the user input.

34. A method according to claim 23, comprising determining an association by querying a database.

35. A method according to claim 34, wherein the database comprises association data curated by a user.

36. A method according to claim 34, wherein the database comprises association data generated based on a machine learning prediction.

37. A method according to claim 34, wherein the database comprises association data generated based on a co-occurrence in literature of the biological entity represented in the user input and a biological entity of the set of biological entities, the co-occurrence being detected by a natural language processing tool.

38. A method according to claim 23, comprising determining an association by causing a machine learning algorithm to generate a prediction.

39. A method according to claim 23, comprising determining an association by causing a natural language processing tool to detect at least one co-occurrence in literature of the biological entity represented in the user input and a biological entity of the set of biological entities.

40. A method according to claim 23, comprising rendering a visual indication of an evidence type of an association.

41. A method according to claim 40, wherein the evidence type comprises human curation, machine learning prediction, or natural language processing.

42. A method according to claim 41, wherein the evidence type comprises machine learning predication and the system comprises a filter module configured to enable the user to filter search results by setting a confidence score range of the machine learning prediction.

43. A method according to claim 41, wherein the evidence type comprises natural language processing and the system comprises a filter module configured to enable the user to filter search results by setting a quantitative natural language processing evidence range.

44. A method according to claim 23, comprising enabling a user to ring fence an area of the visualisation and to generate notifications when there are new associations or upgraded evidence types for associations in the ring-fenced area.

45. A system for searching a set of entities, the system comprising:
a user input module configured to receive a user input comprising a representation of an entity;
a search module configured to determine which entities of a set of entities are associated with the user input;
a visualisation module configured to render a visualisation of multiple entities of the set and of parent-child relationships between them; and
an overlay module configured to render an association indicator visually indicating one or more entities of the visualisation that are associated with the user input.
